(19) United States
(12) Patent Application Publication
(10) Pub. No.: US 2026/0195897 A1
(43) Pub. Date: Jul. 9, 2026

(54) MULTI-SCALE FOUNDATION MODEL FOR PREDICTING PROSTATE CANCER PROGRESSION USING LONGITUDINAL MRI IMAGES

(71) Applicant: Siemens Healthineers AG, Forchheim (DE)

(72) Inventors: Bin Lou, Princeton Junction, NJ (US); Ali Kamen, Skillman, NJ (US)

(21) Appl. No.: 19/015,380

(22) Filed: Jan. 9, 2025

(51) Int. Cl. G06T 7/00; A61B 5/00; A61B 5/055; G06T 7/11; G06T 7/174; G06T 11/00; G06V 10/26; G06V 10/62; G06V 10/77; G06V 10/82

(52) U.S. Cl. CPC G06T 7/0016; A61B 5/055; A61B 5/4381; A61B 5/4842; A61B 5/7267; A61B 5/7275; G06T 7/11; G06T 7/174; G06T 12/20; G06V 10/26; G06V 10/62; G06V 10/7715; G06V 10/82; G06T 2200/24; G06T 2207/10088; G06T 2207/20081; G06T 2207/20084; G06T 2207/30081; G06T 2207/30096; G06T 2210/41; G06T 2211/441; G06V 2201/03

(57) ABSTRACT

Systems and methods for evaluating progression of an anatomical object over a plurality of timepoints are provided. Longitudinal medical images of an anatomical object of a patient acquired over a plurality of timepoints are received. For each respective timepoint of the plurality of timepoints, features are extracted from the longitudinal medical images acquired at the respective timepoint using a machine learning based feature extractor network and the anatomical object in the longitudinal medical images acquired at the respective timepoint is analyzed based on the extracted features using a machine learning based prediction model. Progression of the anatomical object over the plurality of timepoints is evaluated based on results of the analyses using a machine learning based progression model. The evaluation of the progression of the anatomical object over the plurality of timepoints is output.

### TECHNICAL FIELD

The present invention relates generally to AI/ML (artificial intelligence/machine learning) based medical imaging analysis, and in particular to a multi-scale foundation model for predicting prostate cancer progression using longitudinal MRI (magnetic resonance imaging) images.

### BACKGROUND

Prostate cancer is one of the most common types of cancers. Nearly half of the patients diagnosed with prostate cancer present with low-risk or favorable intermediate-risk, for which active surveillance is the recommended treatment option. Active surveillance involves regular monitoring of the prostate cancer without immediate treatment. Such monitoring typically involves follow-up examinations with PSA (prostate specific antigen) tests, mp-MRI (multi-parametric magnetic resonance imaging) imaging, and prostate biopsies. When prostate cancer lesions are in their early stages, grading MRI-detected prostate cancer legions and evaluating whether progression has occurred or will occur is a difficult and time-consuming task.

Recently, AI-based computer-aided detection systems have been proposed for the detection and assessment of prostate cancer based on mp-MRI images. However, such conventional AI-based computer-aided detection systems utilize only baseline images and follow-up images from a single follow-up active surveillance examination. Accordingly, such conventional AI-based computer-aided detection systems are unable to utilize follow-up images from prior follow-up active surveillance examinations and provide dynamic updates on progression risk.

### BRIEF SUMMARY OF THE INVENTION

In accordance with one or more embodiments, systems and methods for evaluating progression of an anatomical object over a plurality of timepoints are provided. Longitudinal medical images of an anatomical object of a patient acquired over a plurality of timepoints are received. For each respective timepoint of the plurality of timepoints, features are extracted from the longitudinal medical images acquired at the respective timepoint using a machine learning based feature extractor network and the anatomical object in the longitudinal medical images acquired at the respective timepoint is analyzed based on the extracted features using a machine learning based prediction model. Progression of the anatomical object over the plurality of timepoints is evaluated based on results of the analyses using a machine learning based progression model. The evaluation of the progression of the anatomical object over the plurality of timepoints is output.

In one embodiment, the machine learning based progression model receives as input an output of a second-to-last layer of the machine learning based prediction model for each of the plurality of timepoints and generates as output the evaluation of the progression of the anatomical object over the plurality of timepoints.

In one embodiment, the longitudinal medical images comprise patches of the anatomical object extracted from original medical images acquired over the plurality of timepoints. The patches are extracted from the original medical images by extracting features from the original medical images for each of the plurality of timepoints using the machine learning based feature extractor network; segmenting the anatomical object from the original medical images based on the extracted features using a machine learning based segmentation network; and extracting the patches from the original medical images based on the segmentation.

In one embodiment, the machine learning based feature extractor network is trained by receiving training medical images. The training medical images are degraded by applying one or more transformations. Training features are extracted from the degraded training medical images using the machine learning based feature extractor network. The training medical images are reconstructed based on the extracted training features using a machine learning based decoder network. The machine learning based feature extractor network and the machine learning based decoder network are trained based on a comparison between the training medical images and reconstructed training medical images.

In one embodiment, the machine learning based feature extractor network is trained using multi-scale training medical images.

In one embodiment, the plurality of timepoints comprises a timepoint corresponding to a baseline examination of the one or more anatomical objects, one or more timepoints corresponding to one or more follow-up examinations of the anatomical objects, and a timepoint corresponding to a current examination of the anatomical objects.

In one embodiment, the anatomical object comprises one or more prostate cancer lesions on a prostate of the patient. The machine learning based progression model is trained to determine at least one of a GGG (Gleason grade group) score or a PIRADS (prostate imaging reporting and data system) score.

In one embodiment, the longitudinal medical images comprise medical images of an MRI (magnetic resonance imaging) sequence.

These and other advantages of the invention will be apparent to those of ordinary skill in the art by reference to the following detailed description and the accompanying drawings.

### BRIEF DESCRIPTION OF THE DRAWINGS

FIG. 1 shows a method for evaluating progression of an anatomical object based on longitudinal medical images, in accordance with one or more embodiments;

FIG. 2 shows a workflow for evaluating progression of lesions based on longitudinal MRI (magnetic resonance imaging) medical images, in accordance with one or more embodiments;

FIG. 3 shows a network architecture of a ViT (vision transformer) for extracting features from longitudinal medical images, in accordance with one or more embodiments;

FIG. 4 shows a workflow for extracting patches from acquired medical images, in accordance with one or more embodiments;

FIG. 5 shows a workflow for training a foundation model encoder for extracting features from medical images, in accordance with one or more embodiments;

FIG. 6 shows a workflow for training a slice level lesion segmentation model, in accordance with one or more embodiments;

FIG. 7 shows a workflow for training a patch level prediction model, in accordance with one or more embodiments;

FIG. 8 shows a workflow for training an activation surveillance model, in accordance with one or more embodiments;

FIG. 9 shows an exemplary artificial neural network that may be used to implement one or more embodiments;

FIG. 10 shows a convolutional neural network that may be used to implement one or more embodiments; and

FIG. 11 shows a high-level block diagram of a computer that may be used to implement one or more embodiments.

### DETAILED DESCRIPTION

The present invention generally relates to methods and systems for predicting prostate cancer progression using longitudinal MRI images. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system. Further, reference herein to pixels of an image may refer equally to voxels of an image and vice versa.

Embodiments described herein provide for a framework for the detection and evaluation of prostate cancer lesions using longitudinal MRI images of a patient. Longitudinal features are extracted from the longitudinal MRI images acquired over a plurality of timepoints using a multi-scale foundation model. The anatomical object in the longitudinal medical images acquired at each respective timepoint is analyzed using a machine learning based prediction model. Progression of the anatomical object over the plurality of timepoints is evaluated based on results of the analyses using a machine learning based progression model. Advantageously, the framework in accordance with embodiments described herein efficiently integrates MRI images acquired over any number of timepoints, thereby resulting in improved diagnostic accuracy of prostate cancer grading and progression prediction as compared to conventional approaches.

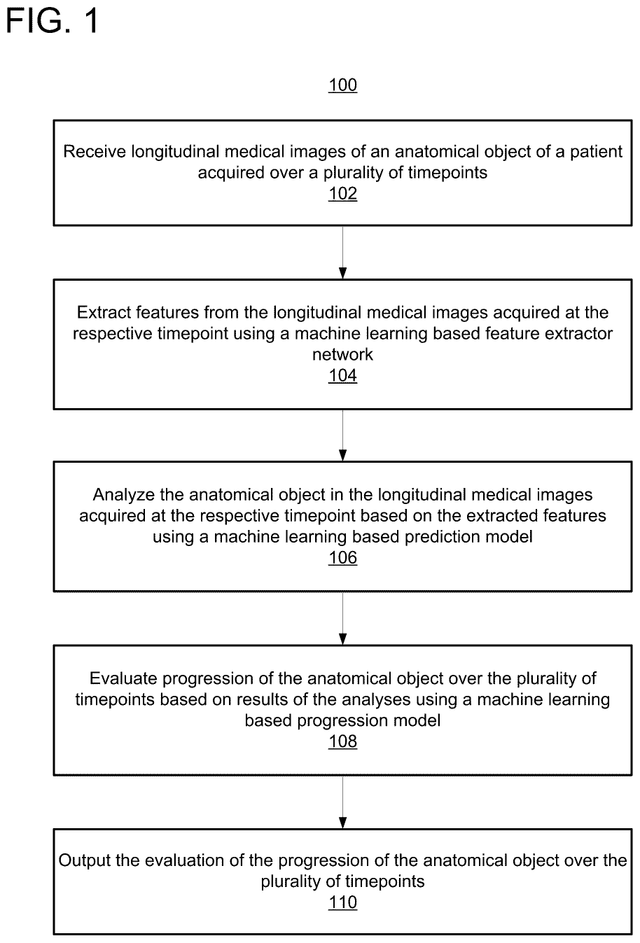

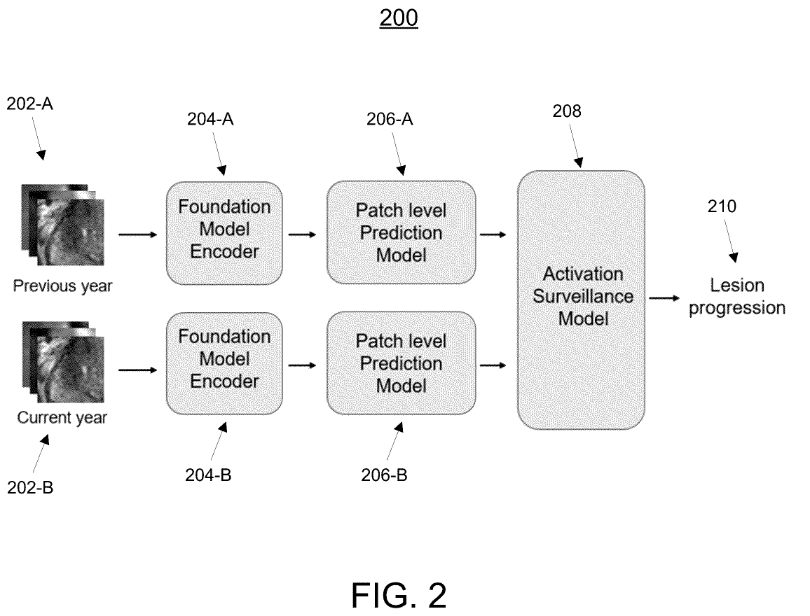

FIG. 1 shows a method 100 for evaluating progression of an anatomical object based on longitudinal medical images, in accordance with one or more embodiments. The steps and sub-steps of method 100 may be performed by one or more suitable computing devices, such as, e.g., computer 1202 of FIG. 12. FIG. 2 shows a workflow 200 for evaluating progression of lesions based on longitudinal MRI medical images, in accordance with one or more embodiments. FIG. 1 and FIG. 2 will be described together.

At step 102 of FIG. 1, longitudinal medical images of an anatomical object of a patient acquired over a plurality of timepoints are received. The plurality of timepoints may correspond to, for example, different examinations (e.g., an initial examination, one or more follow-up examinations, and a current/most recent examination) or different years (e.g., a current year and previously years). In one example, as shown in workflow 200 of FIG. 2, the longitudinal medical images are medical images 202-A and 202-B (collectively referred to as longitudinal medical images 202) respectively acquired for a previous year and a current year.

In one embodiment, the anatomical object comprises one or more prostate cancer lesions (e.g., prostate cancer lesions) on a prostate of the patient. However, the anatomical object may comprise any other suitable anatomical object or objects of interest, such as, e.g., tumors or other abnormalities, organs, bones, vessels, etc. The longitudinal medical images may have been acquired for monitoring progression of the anatomical object over the plurality of timepoints, such as, e.g., to monitor progression of the prostate cancer lesions during active surveillance.

In one embodiment, the longitudinal medical images comprise medical images of an MRI (magnetic resonance imaging) sequence, such as, e.g., T2-weighted images, ADC (apparent diffusion coefficient) maps, DWI (diffusion-weighted imaging) images, etc. However, the longitudinal medical images may comprise medical images of any other suitable domain or domains. As used herein, a domain of a medical image refers to the modality of the medical image as well as the protocol used for obtaining the medical image in that modality. The modality of the medical images may include, for example, MRI, CT (computed tomography), US (ultrasound), x-ray, SPECT (single-photon emission computed tomography), PET (positron emission tomography), or any other medical imaging modality or combinations of medical imaging modalities. The protocol used for obtaining the medical image may include, for example, acquisition sequences or techniques for acquiring a medical image, such as, e.g., T1-weighted, T2-weighted, proton density-weighted MRI images, contrast and non-contrast images, CT images captured with low kV (kilovoltage) and high kV, or low- and high-resolution medical images. Accordingly, the domains may be completely different medical imaging modalities or different image protocols within the same overall imaging modality. The longitudinal medical images may comprise 2D (two dimensional) images and/or 3D (three dimensional) volumes.

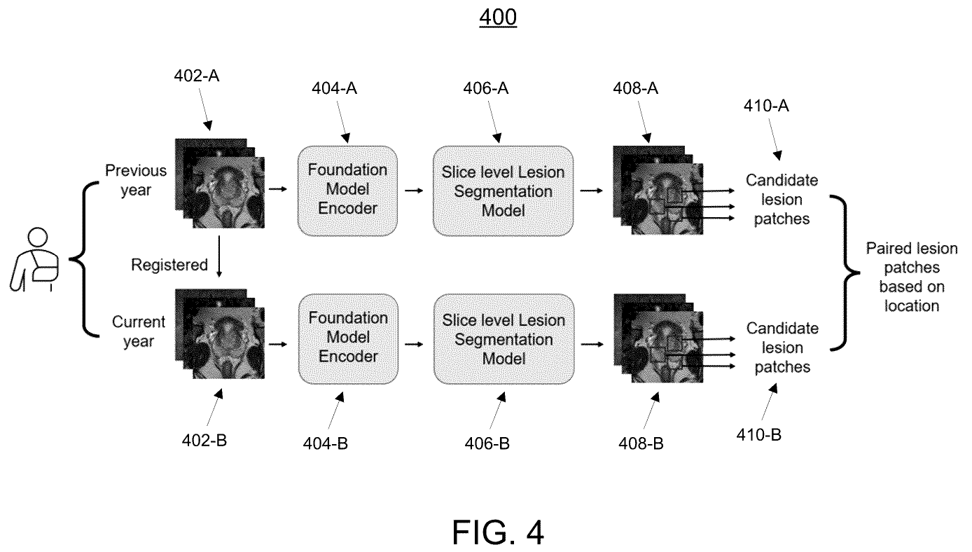

In one embodiment, the longitudinal medical images comprise patches of the anatomical object. The patches may be extracted from acquired original medical images, for example, according to workflow 400 of FIG. 4, described in detail below.

The longitudinal medical images may be received, for example, by directly receiving the longitudinal medical images from an image acquisition device (e.g., image acquisition device 1214 of FIG. 12) as the images are acquired, by loading the longitudinal medical images from a storage or memory of a computer system (e.g., storage 1212 or memory 1210 of computer 1202 of FIG. 12), or by receiving the longitudinal medical images from a remote computer system (e.g., computer 1202 of FIG. 12). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

Steps 104 and 106 of FIG. 1 are performed for each respective timepoint of the plurality of timepoints.

At step 104 of FIG. 1, features are extracted from the longitudinal medical images acquired at the respective timepoint using a machine learning based feature extractor network. For example, as shown in workflow 200 of FIG. 2, features are respectively extracted from longitudinal medical images 202-A and 202-B using foundation model encoder 204-A and 204-B (collectively referred to as foundation model encoder 204). While foundation model encoders 204-A and 204-B are separately shown in workflow 200 to illustrate processing of longitudinal medical images 202-A and 202-B, it should be understood that foundation model encoders 204-A and 204-B are the same foundation model encoder.

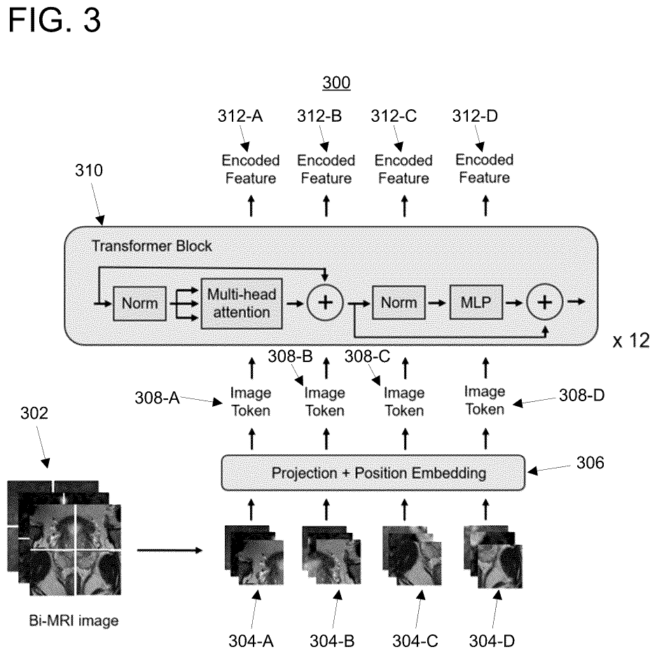

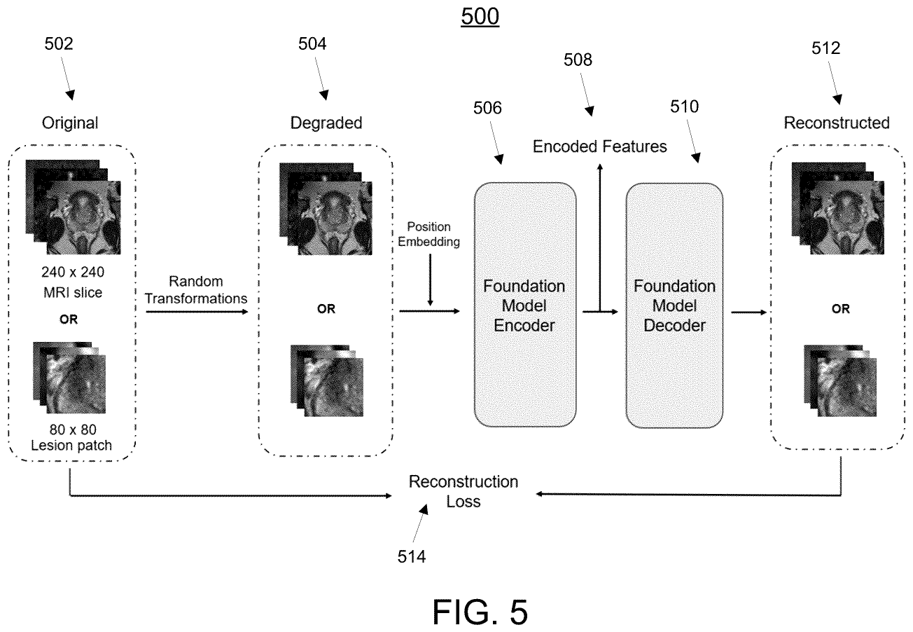

In one embodiment, the machine learning based feature extractor network is a foundation model, such as, e.g., a ViT (vision transformer). For example, the ViT may be implemented according to network architecture 300 of FIG. 3, described in detail below. However, the machine learning based feature extractor network may be implemented according to any other suitable machine learning based architecture. The machine learning based feature extractor network receives as input longitudinal medical images for the respective timepoint and generates as output features for that respective timepoint. Accordingly, the features for the plurality of timepoints are longitudinal features. Features are low-level latent representations or embeddings of the images. The machine learning based feature extractor network is trained during a prior offline or training stage, for example, according to workflow 500 of FIG. 5, as discussed in detail below. The machine learning based feature extractor network is trained to extract features using, e.g., multi-scale training medical images (e.g., original images and patches extracted therefrom). Once trained, the machine learning based feature extractor network is applied during an online or inference stage, e.g., to perform step 104 of FIG. 1.

FIG. 3 shows a network architecture 300 of a ViT for extracting features from longitudinal medical images, in accordance with one or more embodiments. In one embodiment, the machine learning based feature extractor network utilized at step 104 of FIG. 1 and the foundation model encoder 204 of FIG. 2 may be implemented according to network architecture 300. As shown in network architecture 300, bi-MRI images 302 of prostate cancer lesions are cropped into patches 304-A, 304-B, 304-C, and 304-D (collectively referred to as patches 304). Patches 304 are input to projection and position embedding layer 306, which projects patches 304 into image tokens 308-A, 308-B, 308-C, and 308-D (collectively referred to as image tokens 308) having a higher-dimensional space and encoded with positional embeddings reflecting the position of the patches 304 in the bi-MRI images 302. Transformer block 310 receives as input image tokens 308-A, 308-B, 308-C, and 308-C and respectively generates as output encoded features 312-A, 312-B, 312-C, and 312-D. As shown in network architecture 300, transformer block 310 comprises normalization layers, a multi-head attention layer, and an MLP (multilayer perceptron) layer.

Returning back to FIG. 1, at step 106, the anatomical object in the longitudinal medical images acquired at the respective timepoint is analyzed based on the extracted features using a machine learning based prediction network. In one example, as shown in workflow 200 of FIG. 2, the prostate cancer lesions in longitudinal medical images 202-A and 202-B acquired at each respective timepoint is analyzed based on features extracted by foundation model encoder 204-A and 204-B using patch level prediction models 206-A and 206-B (collectively referred to patch level prediction models 206). While patch level prediction models 206-A and 206-B are separately shown in workflow 200 to illustrate processing of longitudinal medical images 202-A and 202-B, it should be understood that patch level prediction models 206-A and 206-B are the same patch level prediction model.

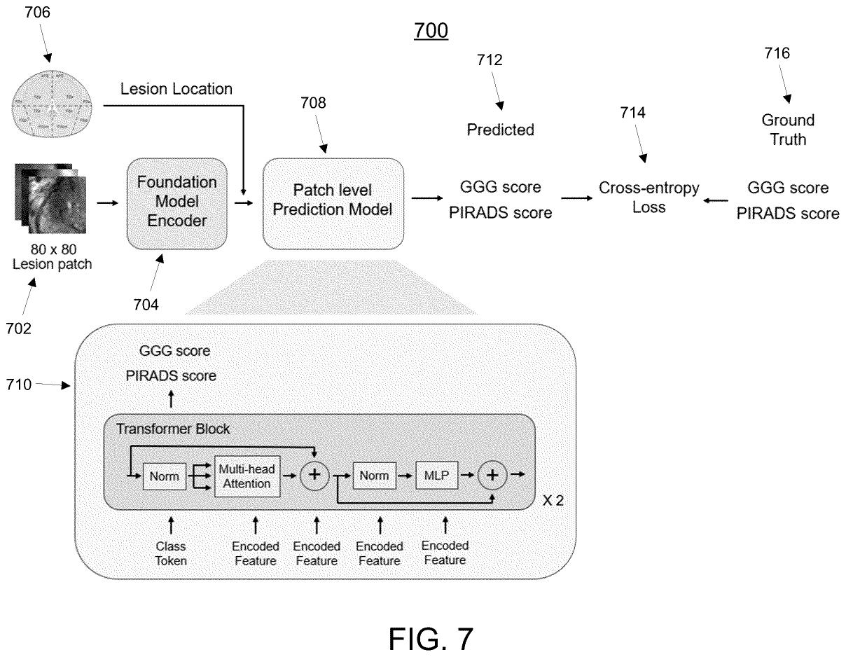

In one embodiment, the machine learning based prediction network comprises a transformer network. However, the machine learning based task network may be implemented according to any other suitable machine learning based architecture. The machine learning based prediction network receives as input the extracted features and generates as output results of the analysis. While the machine learning based prediction network is trained to predict a score associated with the anatomical object (e.g., a GGG (Gleason grade group) score and/or a PIRADS (prostate imaging-reporting and data system) score), the results of the analysis to be determined at step 106 and utilized at step 108 is the output of the second-to-last layer of the machine learning based prediction network. The output of the second-to-last layer of the machine learning based prediction network is a high-dimensional vector that includes high-level information of the content of the longitudinal medical images acquired at the respective timepoint. The machine learning based prediction network is trained during a prior offline or training stage, for example, according to workflow 700 of FIG. 7, as discussed in detail below. Once trained, the machine learning based feature extractor network is applied during an online or inference stage, e.g., to perform step 106 of FIG. 1.

At step 108 of FIG. 1, progression of the anatomical object over the plurality of timepoints is evaluated based on results of the analyses using a machine learning based progression model. In one example, as shown in workflow 200 of FIG. 2, lesion progression 210 is determined by activation surveillance model 208 based on the output of the second-to-last layer of patch level prediction models 206.

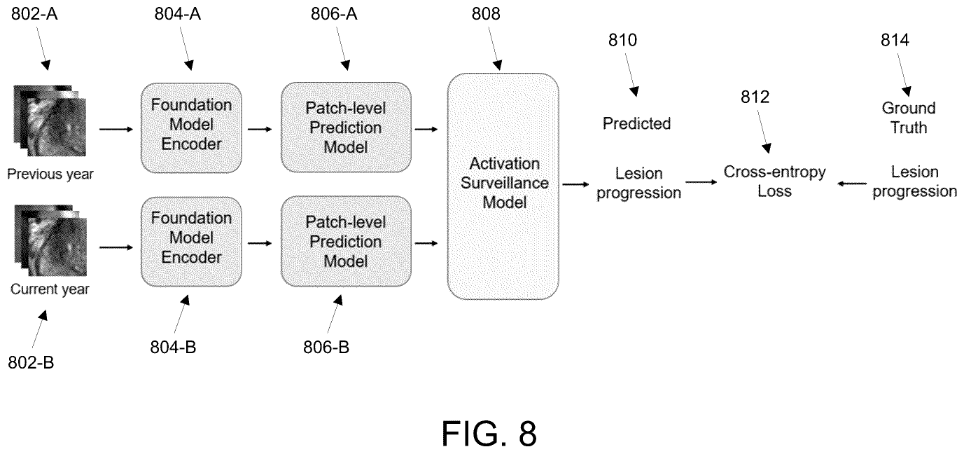

In one embodiment, the machine learning based progression network comprises a transformer network. However, the machine learning based progression network may be implemented according to any other suitable machine learning based architecture. The machine learning based progression network receives as input the results of the analyses (i.e., the output of the second-to-last layer of the machine learning based prediction network) and generates as output a prediction of the progression of the anatomical objection. The progression may be represented in any suitable format, such as, e.g., a classification (e.g., progressed or not progressed), a progression score, etc. The machine learning based progression network is trained during a prior offline or training stage, for example, according to workflow 800 of FIG. 8, as discussed in detail below. Once trained, the machine learning based feature extractor network is applied during an online or inference stage, e.g., to perform step 108 of FIG. 1.

At step 110 of FIG. 1, the evaluation of the progression of the anatomical object over the plurality of timepoints is output. For example, the evaluation of the progression of the anatomical object over the plurality of timepoints can be output by displaying the evaluation on a display device of a computer system (e.g., I/O 1208 of computer 1202 of FIG. 12), storing the results on a memory or storage of a computer system (e.g., memory 1210 or storage 1212 of computer 1202 of FIG. 12), or by transmitting the results to a remote computer system (e.g., computer 1202 of FIG. 12).

FIG. 4 shows a workflow 400 for extracting patches from acquired medical images, in accordance with one or more embodiments. The patches extracted in accordance with workflow 400 may be the longitudinal medical images received at step 102 of FIG. 1.

In workflow 400, original longitudinal medical images 402-A and 402-B (collectively referred to as longitudinal medical images 402) of an anatomical object of a patient acquired over a plurality of timepoints are received. In one embodiment, longitudinal medical images 402 may comprise medical images of an MRI sequence. However, longitudinal medical images 402 may be of any other suitable domain or domains. As shown in workflow 400, longitudinal medical images 402-A and 402-B are acquired at timepoints corresponding to a previous year and a current year respectively. Longitudinal medical images 402 are registered (e.g., according to any well-known image registration technique)

Longitudinal medical images 402 may be received, for example, by directly receiving the longitudinal medical images from an image acquisition device (e.g., image acquisition device 1214 of FIG. 12) as the images are acquired, by loading the longitudinal medical images from a storage or memory of a computer system (e.g., storage 1212 or memory 1210 of computer 1202 of FIG. 12), or by receiving the longitudinal medical images from a remote computer system (e.g., computer 1202 of FIG. 12).

Features are extracted from longitudinal medical images 402-A and 402-B using foundation model encoder 404-A and 404-B (collectively referred to as foundation model encoder 404) respectively. While foundation model encoders 404-A and 404-B are separately shown in workflow 400 to illustrate processing of longitudinal medical images 402-A and 402-B, it should be understood that foundation model encoders 404-A and 404-B are the same foundation model encoder. Foundation model encoder 404 may be the machine learning based feature extractor network utilized at step 104 of FIG. 1 or foundation model encoder 204 of FIG. 2. Foundation model encoder 404 may be implemented as a ViT or with any other suitable machine learning based architecture. Foundation model encoder 404 respectively receives as input longitudinal medical images 402 for the respective timepoint and generates as output features for that respective timepoint. Foundation model encoder 404 is trained during a prior offline or training stage, for example, according to workflow 500 of FIG. 5, as discussed in detail below. Once trained, foundation model encoder 404 is applied during an online or inference stage, e.g., to extract features from longitudinal medical images 402 in workflow 400 of FIG. 4.

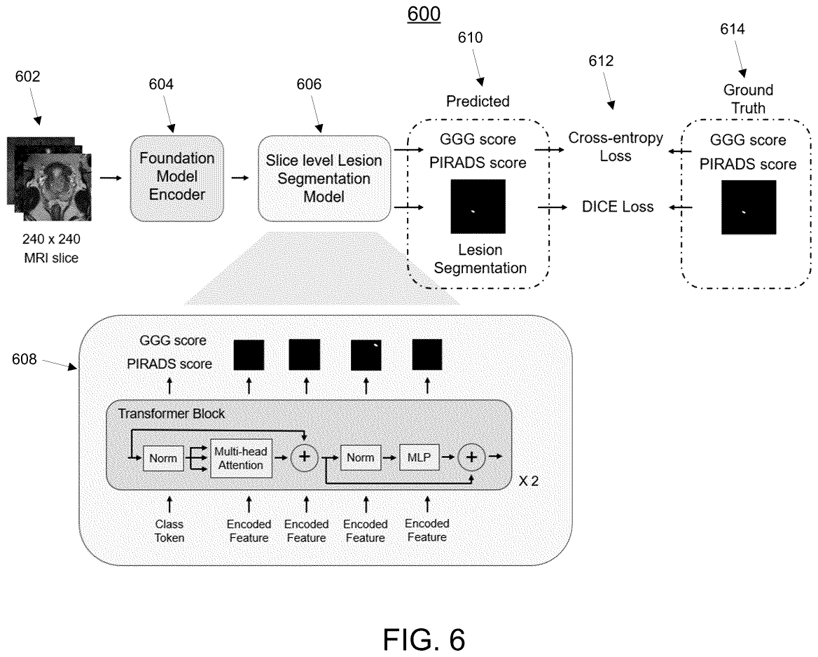

The anatomical object is segmented from longitudinal medical images 402-A and 402-B using slice level lesion segmentation models 406-A and 406-B (collectively referred to as segmentation model 406) respectively. While slice level lesion segmentation models 406-A and 406-B are separately shown in workflow 400 to illustrate processing of longitudinal medical images 402-A and 402-B, it should be understood that slice level lesion segmentation models 406-A and 406-B are the same segmentation model. In one embodiment, slice level lesion segmentation model is implemented using a transformer network, but may be implemented using any other suitable machine learning based architecture. Slice level lesion segmentation models 406-A and 406-B respectively receive as input features extracted from longitudinal medical images 402-A and 402-B by foundation model encoders 404-A and 404-B and generates as output segmentation maps 408-A and 408-B (collectively referred to as segmentation maps 408). Segmentation maps 408 identify candidate lesions (or any other anatomical objects). Patches of the identified candidate lesions are then respectively extracted from longitudinal medical images 402-A and 402-B based on the segmentation maps 408-A and 408-B to provide for paired candidate lesion patches 410-A and 410B. Segmentation model 406 is trained during a prior offline or training stage, for example, according to workflow 600 of FIG. 6, as discussed in detail below. Once trained, segmentation model 406 is applied during an online or inference stage, e.g., to segment the anatomical object from longitudinal medical images 402.

FIGS. 5-8 show training of various machine learning based networks utilized herein. Such machine learning based networks are trained during a prior offline or training stage. Once trained, the machine learning based networks are applied during an online or inference stage, e.g., to perform various steps of method 100 of FIG. 1 and/or workflow 200 of FIG. 2.

FIG. 5 shows a workflow 500 for training a foundation model encoder 506 for extracting features from medical images, in accordance with one or more embodiments. Foundation model encoder 506 may be implemented as a transformer network, e.g., according to network architecture 300 of FIG. 3. Foundation model encoder 506 is trained together with foundation model decoder 510 using self-supervised or unsupervised learning according to workflow 500 during an offline or training stage.

As shown in workflow 500, original training medical images 502 are transformed into degraded images 504. In one embodiment, original training medical images 502 are medical images of an MRI sequence, but may be of any other suitable domain. Original training medical images 502 may comprise an MRI slice or patches extracted therefrom. For example, the MRI slice may first be resampled to dimensions of 240×240 pixels, with a voxel spacing of 0.5 mm×0.5 mm (millimeters), to provide for original training medical images 502. In another example, the patches may have a size of 80×80 pixels randomly cropped from MRI slices to provide for original training medical images 502. Original training medical images 502 may be randomly transformed by, e.g., non-linear pixel value adjustments, pixel shuffling, application of small random patch masks, or any other suitable transformation technique. Degraded images 504 may be encoded with position embedding representing the location of the patches.

Foundation model encoder 506 receives as input degraded images 504 and generates as output encoded features 508. Foundation model decoder 510 decodes encoded features 508 to generate reconstructed images 512 representing a reconstruction of original training medical images 502. Foundation model encoder 506 is trained with foundation model decoder 510 by comparing original training medical images 502 with reconstructed images 512 according to a reconstruction loss function 514, such as, e.g., MSE (mean squared errors). Foundation model encoder 506 is thus trained to capture structural characteristics of the anatomical object and radiomic features and learn a common image representation that is both transferable and generalizable. After training, foundation model encoder 506 is applied during an online or inference stage, e.g., as the machine learning based feature extractor network utilized at step 104 of FIG. 1, foundation model encoder 204 of FIG. 2, or foundation model encoder 404 of FIG. 4. Foundation model decoder 510 is not utilized during inference. By training foundation model encoder 506 using both slices and patches, foundation model encoder 506 trained to handle multi-scale images for feature extraction.

FIG. 6 shows a workflow 600 for training a slice level lesion segmentation model 606, in accordance with one or more embodiments. Training medical images 602 of an anatomical object are first received, which are 240×240 MRI slices in workflow 600. Features are extracted from training medical images 602 by foundation model encoder 604. Foundation model encoder 604 may be trained according to workflow 500 of FIG. 5 during a prior offline or training stage. The weights of foundation model encoder 604 are frozen during workflow 600.

Slice level lesion segmentation model 606 segments the anatomical object from training medical images 602. Slice level lesion segmentation model 606 receives as input the features extracted by foundation model encoder 604 and generates as output predicted results 610 comprising a segmentation map of the anatomical object in the training medical images 602 and a grading of the anatomical object. Segmentation model 606 may be implemented using a transformer block, but may be implemented according to any other suitable machine learning based architecture. Block 608 shows an exploded view of slice level lesion segmentation model 606. As shown in block 608, transformer block comprises normalization layers, multi-head attention layer, and a MLP layer. The transformer block receives as input encoded features and a class token and generates as output segmentation maps and a grading (e.g., GGG score and PIRADS score). Predicted results 610 are compared with ground truth results 614 according to loss functions 612, such as, e.g., a cross-entropy loss and a Dice loss. Once trained, slice level lesion segmentation model 606 may applied during an online or inference stage, e.g., as slice level lesion segmentation model 406 of FIG. 4.

FIG. 7 shows a workflow 700 for training a patch level prediction model 708, in accordance with one or more embodiments. Training medical images 702 of are first received, which are 80×80 patches. Training medical images 702 may depict a lesion (or any other anatomical object) with either a GGG score greater than 0 or a PIRADS score of 3 or higher. Additionally, training medical images 702 may be randomly cropped image patches without lesions to serve as negative samples. Features are extracted from training medical images 702 by foundation model encoder 704. Foundation model encoder 804 may be trained during a prior offline or training stage according to workflow 500 of FIG. 5. The weights of foundation model encoder 704 are frozen during workflow 700. The features are encoded with lesion location 706. Such lesions may be detected by, for example, the slice level lesion segmentation model trained according to workflow 600 of FIG. 6.

Patch level prediction model 708 predicts an analysis of the lesion in training medical images 702. Patch level prediction model 708 receives as input the features extracted by foundation model encoder 704 and generates as output predicted results of 712 of the analysis comprising, e.g., a GGG score and a PIRADS score. Patch level prediction model 708 may be implemented using a transformer block, but may be implemented according to any other suitable machine learning based architecture. Block 710 shows an exploded view of patch level prediction model 708. As shown in block 710, transformer block comprises normalization layers, multi-head attention layer, and a MLP layer. The transformer block receives as input encoded features and a class token and generates as output a grading (e.g., GGG score and PIRADS score) of the lesion. Predicted results 712 are compared with ground truth results 716 according to loss function 714, such as, e.g., a cross-entropy loss. Once trained, patch level prediction model 708 may applied during an online or inference stage, e.g., as machine learning based prediction model utilized at step 106 of FIG. 1 or patch level prediction model 206 of FIG. 2.

FIG. 8 shows a workflow 800 for training an activation surveillance model 808, in accordance with one or more embodiments. Longitudinal training medical images 802-A and 802-B (collectively referred to as longitudinal training medical images 802) acquired over a plurality of timepoints are first received. Due to the limited availability of paired lesion data (lesion patch images from both the previous and current year), activation surveillance model 808 is pre-trained using synthetic paired lesion data. Regions of interest identified by users as positive lesions are extracted and treated as the lesion patch for the current year. This will be paired with an image patch from the same anatomical location of a different patient without positive prostate lesion (after registration between the two patients) to serve as the previous year's lesion patch, simulating a “progressed” lesion scenario. Additionally, image patches from two patients, either both with or both without lesions in the same location after registration, will are selected to form non-progression training pairs.

Features are respectively extracted from longitudinal training medical images 802-A and 80-B by foundation model encoders 804-A and 804-B (collectively referred to as foundation model encoders 804). Analysis of the lesions is performed by patch-level prediction models 806-A and 806-B (collectively referred to as patch-level prediction model 806). While foundation model encoders 804-A and 804-B and patch-level prediction models 806-A and 806-B are separately shown in workflow 800 to illustrate processing of longitudinal training medical images 202-A and 202-B, it should be understood that foundation model encoders 204-A and 204-B and patch-level prediction models 806-A and 806-B are the same foundation model encoder and the same patch-level prediction model, respectively. Foundation model encoder 804 may be trained during a prior offline or training stage according to workflow 500 of FIG. 5 and patch-level prediction model 806 may be trained during a prior offline or training stage according to workflow 700 of FIG. 7. The weights of foundation model encoder 804 and patch-level prediction model 806 are frozen during workflow 800.

Activation surveillance model predicts an evaluation of progression of the lesion. Activation surveillance model 808 receives as input the output of the second-to-last layer of patch-level prediction model 806 and generates as output predicted results 810 of the evaluation of lesion progression. Activation surveillance model 808 may be implemented using a transformer block, but may be implemented according to any other suitable machine learning based architecture. Predicted results 810 are compared with ground truth results 814 according to loss function 812, such as, e.g., a cross-entropy loss. Once trained, activation surveillance model 808 may applied during an online or inference stage, e.g., as machine learning based progression model utilized at step 108 of FIG. 1 or activation surveillance model 208 of FIG. 2.

Advantageously, embodiments described herein efficient integrate medical images from previous years/examinations to improve the accuracy of prostate lesion detection and risk assessment in the current year/examination. By leveraging a multi-scale foundation model and multi-step transfer learning, embodiments described herein effectively utilizes a large dataset of images from a single-timepoint prostate examination to address challenges in active surveillance. Embodiments described herein are capable of managing the entire active surveillance diagnostic pipeline independently, without relying on additional models.

Further, embodiments described herein utilize self-supervised techniques to develop the multi-scale foundation model, which can leverage extensive individual single-time prostate MRI images to improve the model's effectiveness in addressing longitudinal prostate cancer active surveillance challenges. Embodiments described herein employ multi-step transfer learning techniques to enable the model to perform various tasks, such as, e.g., prostate lesion segmentation, lesion grading, and disease progression prediction in an active surveillance setting. Additionally, throughout the transfer learning process, the model incrementally refines its understanding of high-level harder tasks, even with a limited dataset.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, a machine learning model, such as, e.g., the machine learning based feature extractor network utilized at step 104, the machine learning based prediction model utilized at step 106, and the machine learning based progression model utilized at step 108 of FIG. 1, foundation model encoder 204, patch level prediction model 206, and activation surveillance model 208 of FIG. 2, transformer block 310 of FIG. 3, foundation model encoder 404 and slice level lesion segmentation model 406 of FIG. 4, foundation model encoder 506 and foundation model decoder 510 of FIG. 5, foundation model encoder 604 and slice level lesion segmentation model 606 of FIG. 6, foundation model encoder 704 and patch level prediction model 708 of FIG. 7, and foundation model encoder 804, patch-level prediction model 806, and activation surveillance model 808 of FIG. 8, can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

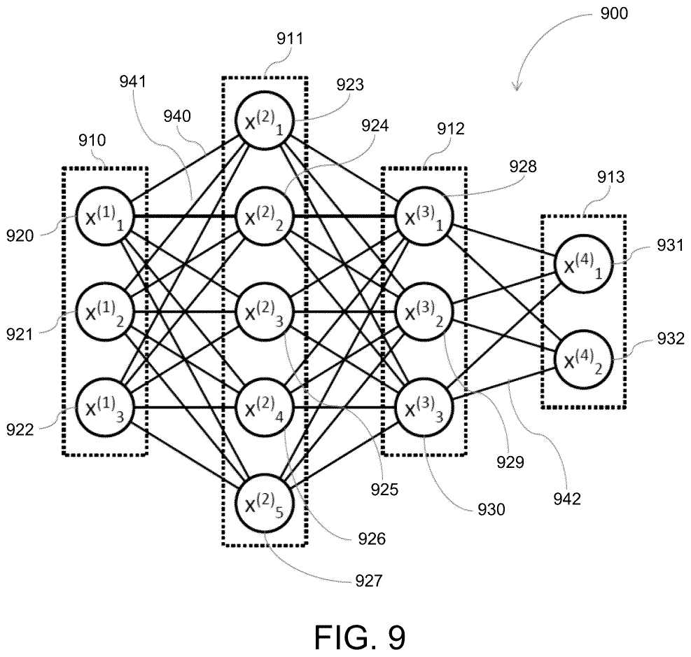

FIG. 9 shows an embodiment of an artificial neural network 900 that may be used to implement one or more machine learning models described herein. Alternative terms for “artificial neural network” are “neural network”, “artificial neural net” or “neural net”.

The artificial neural network 900 comprises nodes 920, . . . , 932 and edges 940, . . . 942, wherein each edge 940, . . . , 942 is a directed connection from a first node 920, . . . , 932 to a second node 920, . . . , 932. In general, the first node 920, . . . , 932 and the second node 920, . . . , 932 are different nodes 920, . . . , 932, it is also possible that the first node 920, . . . , 932 and the second node 920, . . . , 932 are identical. For example, in FIG. 9 the edge 940 is a directed connection from the node 920 to the node 923, and the edge 942 is a directed connection from the node 930 to the node 932. An edge 940, . . . , 942 from a first node 920, . . . , 932 to a second node 920, . . . , 932 is also denoted as “ingoing edge” for the second node 920, . . . , 932 and as “outgoing edge” for the first node 920, . . . , 932.

In this embodiment, the nodes 920, . . . , 932 of the artificial neural network 900 can be arranged in layers 910, . . . , 913, wherein the layers can comprise an intrinsic order introduced by the edges 940, . . . , 942 between the nodes 920, . . . , 932. In particular, edges 940, . . . , 942 can exist only between neighboring layers of nodes. In the displayed embodiment, there is an input layer 910 comprising only nodes 920, . . . , 922 without an incoming edge, an output layer 913 comprising only nodes 931, 932 without outgoing edges, and hidden layers 911, 912 in-between the input layer 910 and the output layer 913. In general, the number of hidden layers 911, 912 can be chosen arbitrarily. The number of nodes 920, . . . , 922 within the input layer 910 usually relates to the number of input values of the neural network, and the number of nodes 931, 932 within the output layer 913 usually relates to the number of output values of the neural network.

In particular, a (real) number can be assigned as a value to every node 920, . . . , 932 of the neural network 900. Here, x⁽ⁿ⁾ᵢ denotes the value of the i-th node 920, 932 of the n-th layer 910, . . . , 913. The values of the nodes 920, . . . , 922 of the input layer 910 are equivalent to the input values of the neural network 900, the values of the nodes 931, 932 of the output layer 913 are equivalent to the output value of the neural network 900. Furthermore, each edge 940, . . . , 942 can comprise a weight being a real number, in particular, the weight is a real number within the interval [−1, 1] or within the interval [0, 1]. Here, w^{(m,n)}_{i,j} denotes the weight of the edge between the i-th node 920, . . . , 932 of the m-th layer 910, . . . , 913 and the j-th node 920, . . . , 932 of the n-th layer 910, . . . , 913. Furthermore, the abbreviation w⁽ⁿ⁾_{i,j} is defined for the weight w^{(n,n+1)}_{i,j}.

In particular, to calculate the output values of the neural network 900, the input values are propagated through the neural network. In particular, the values of the nodes 920, . . . , 932 of the (n+1)-th layer 910, . . . , 913 can be calculated based on the values of the nodes 920, . . . , 932 of the n-th layer 910, . . . , 913 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 910 are given by the input of the neural network 900, wherein values of the first hid-den layer 911 can be calculated based on the values of the input layer 910 of the neural network, wherein values of the second hidden layer 912 can be calculated based in the values of the first hidden layer 911, etc.

In order to set the values w^{(m,n)}_{i,j} for the edges, the neural network 900 has to be trained using training data. In particular, training data comprises training input data and training output data (denoted as tᵢ). For a training step, the neural network 900 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 900 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾ⱼ can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾ⱼ, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i , j}} )$$

if the (n+1)-th layer is the output layer 913, wherein f′ is the first derivative of the activation function, and t⁽ⁿ⁺¹⁾ⱼ is the comparison training value for the j-th node of the output layer 913.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

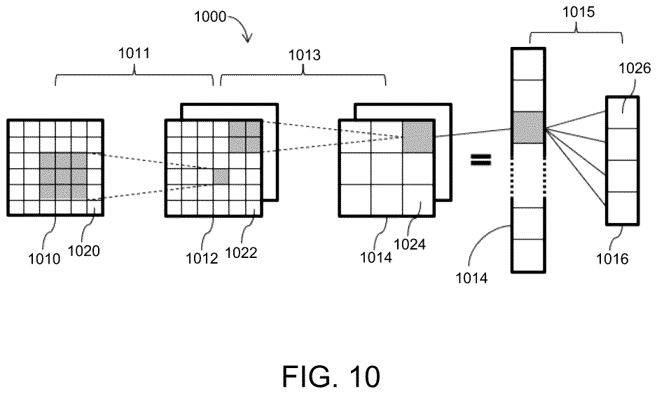

FIG. 10 shows an embodiment of a convolutional neural network 1000 that may be used to implement one or more machine learning models described herein. In the displayed embodiment, the convolutional neural network 1000 comprises an input node layer 1010, a convolutional layer 1011, a pooling layer 1013, a fully connected layer 1014 and an output node layer 1016, as well as hidden node layers 1012, 1014. Alternatively, the convolutional neural network 1000 can comprise several convolutional layers 1011, several pooling layers 1013 and several fully connected layers 1015, as well as other types of layers. The order of the layers can be chosen arbitrarily, usually fully connected layers 1015 are used as the last layers before the output layer 1016.

In particular, within a convolutional neural network 1000 nodes 1020, 1022, 1024 of a node layer 1010, 1012, 1014 can be considered to be arranged as a d-dimensional matrix or as a d-dimensional image. In particular, in the two-dimensional case the value of the node 1020, 1022, 1024 indexed with i and j in the n-th node layer 1010, 1012, 1014 can be denoted as x(n)[i, j]. However, the arrangement of the nodes 1020, 1022, 1024 of one node layer 1010, 1012, 1014 does not have an effect on the calculations executed within the convolutional neural network 1000 as such, since these are given solely by the structure and the weights of the edges.

A convolutional layer 1011 is a connection layer between an anterior node layer 1010 (with node values x(n−1)) and a posterior node layer 1012 (with node values x(n)). In particular, a convolutional layer 1011 is characterized by the structure and the weights of the incoming edges forming a convolution operation based on a certain number of kernels. In particular, the structure and the weights of the edges of the convolutional layer 1011 are chosen such that the values x(n) of the nodes 1022 of the posterior node layer 1012 are calculated as a convolution x(n)=K*x(n−1) based on the values x(n−1) of the nodes 1020 anterior node layer 1010, where the convolution * is defined in the two-dimensional case as

$${x}_{k}^{( n )} [ i , j ] = ( K * {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , j - {j}^{′} ] .$$

Here the kernel K is a d-dimensional matrix (in this embodiment, a two-dimensional matrix), which is usually small compared to the number of nodes 1020, 1022 (e.g., a 3×3 matrix, or a 5×5 matrix). In particular, this implies that the weights of the edges in the convolution layer 1011 are not independent, but chosen such that they produce said convolution equation. In particular, for a kernel being a 3×3 matrix, there are only 9 independent weights (each entry of the kernel matrix corresponding to one independent weight), irrespectively of the number of nodes 1020, 1022 in the anterior node layer 1010 and the posterior node layer 1012.

In general, convolutional neural networks 1000 use node layers 1010, 1012, 1014 with a plurality of channels, in particular, due to the use of a plurality of kernels in convolutional layers 1011. In those cases, the node layers can be considered as (d+1)-dimensional matrices (the first dimension indexing the channels). The action of a convolutional layer 1011 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 1010, x⁽ⁿ⁾^{_{b}} corresponds to the b-th channel of the posterior node layer 1012 and K_{a,b} corresponds to one of the kernels. If a convolutional layer 1011 acts on an anterior node layer 1010 with A channels and outputs a posterior node layer 1012 with B channels, there are A·B independent d-dimensional kernels K_{a,b}.

In general, in convolutional neural networks 1000 activation functions are used. In this embodiment ReLU (acronym for “Rectified Linear Units”) is used, with R(z)=max(0, z), so that the action of the convolutional layer 1011 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 1010 comprises 36 nodes 1020, arranged as a two-dimensional 6×6 matrix. The first hidden node layer 1012 comprises 72 nodes 1022, arranged as two two-dimensional 6×6 matrices, each of the two matrices being the result of a convolution of the values of the input layer with a 3×3 kernel within the convolutional layer 1011. Equivalently, the nodes 1022 of the first hidden node layer 1012 can be interpreted as arranged as a three-dimensional 2×6×6 matrix, wherein the first dimension correspond to the channel dimension.

The advantage of using convolutional layers 1011 is that spatially local correlation of the input data can exploited by enforcing a local connectivity pattern between nodes of adjacent layers, in particular by each node being connected to only a small region of the nodes of the preceding layer.

A pooling layer 1013 is a connection layer between an anterior node layer 1012 (with node values x(n−1)) and a posterior node layer 1014 (with node values x(n)). In particular, a pooling layer 1013 can be characterized by the structure and the weights of the edges and the activation function forming a pooling operation based on a non-linear pooling function f. For example, in the two-dimensional case the values x(n) of the nodes 1024 of the posterior node layer 1014 can be calculated based on the values x(n−1) of the nodes 1022 of the anterior node layer 1012 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 1013 the number of nodes 1022, 1024 can be reduced, by re-placing a number d1·d2 of neighboring nodes 1022 in the anterior node layer 1012 with a single node 1022 in the posterior node layer 1014 being calculated as a function of the values of said number of neighboring nodes. In particular, the pooling function f can be the max-function, the average or the L2-Norm. In particular, for a pooling layer 1013 the weights of the incoming edges are fixed and are not modified by training.

The advantage of using a pooling layer 1013 is that the number of nodes 1022, 1024 and the number of parameters is reduced. This leads to the amount of computation in the network being reduced and to a control of overfitting.

In the displayed embodiment, the pooling layer 1013 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 1000 are fully connected layers 1015. A fully connected layer 1015 is a connection layer between an anterior node layer 1014 and a posterior node layer 1016. A fully connected layer 1013 can be characterized by the fact that a majority, in particular, all edges between nodes 1014 of the anterior node layer 1014 and the nodes 1016 of the posterior node layer are present, and wherein the weight of each of these edges can be adjusted individually.

In this embodiment, the nodes 1024 of the anterior node layer 1014 of the fully connected layer 1015 are displayed both as two-dimensional matrices, and additionally as non-related nodes (indicated as a line of nodes, wherein the number of nodes was reduced for a better presentability). This operation is also denoted as “flattening”. In this embodiment, the number of nodes 1026 in the posterior node layer 1016 of the fully connected layer 1015 smaller than the number of nodes 1024 in the anterior node layer 1014. Alternatively, the number of nodes 1026 can be equal or larger.

Furthermore, in this embodiment the Softmax activation function is used within the fully connected layer 1015. By applying the Softmax function, the sum the values of all nodes 1026 of the output layer 1016 is 1, and all values of all nodes 1026 of the output layer 1016 are real numbers between 0 and 1. In particular, if using the convolutional neural network 1000 for categorizing input data, the values of the output layer 1016 can be interpreted as the probability of the input data falling into one of the different categories.

In particular, convolutional neural networks 1000 can be trained based on the backpropagation algorithm. For preventing overfitting, methods of regularization can be used, e.g., dropout of nodes 1020, . . . , 1024, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-8. Certain steps or functions of the methods and workflows described herein, including one or more of the steps or functions of FIGS. 1-8, may be performed by a server or by another processor in a network-based cloud-computing system. Certain steps or functions of the methods and workflows described herein, including one or more of the steps of FIGS. 1-8, may be performed by a client computer in a network-based cloud computing system. The steps or functions of the methods and workflows described herein, including one or more of the steps of FIGS. 1-8, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-8, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

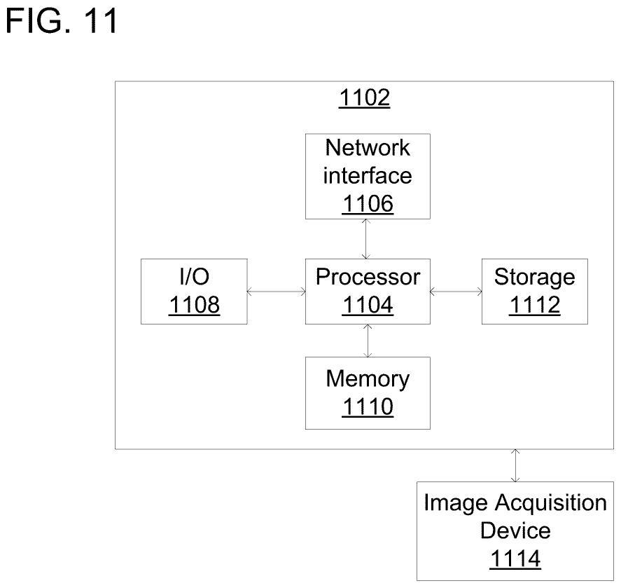

A high-level block diagram of an example computer 1102 that may be used to implement systems, apparatuses, and methods described herein is depicted in FIG. 11. Computer 1102 includes a processor 1104 operatively coupled to a data storage device 1112 and a memory 1110. Processor 1104 controls the overall operation of computer 1102 by executing computer program instructions that define such operations. The computer program instructions may be stored in data storage device 1112, or other computer readable medium, and loaded into memory 1110 when execution of the computer program instructions is desired. Thus, the method and workflow steps or functions of FIGS. 1-8 can be defined by the computer program instructions stored in memory 1110 and/or data storage device 1112 and controlled by processor 1104 executing the computer program instructions. For example, the computer program instructions can be implemented as computer executable code programmed by one skilled in the art to perform the method and workflow steps or functions of FIGS. 1-8. Accordingly, by executing the computer program instructions, the processor 1104 executes the method and workflow steps or functions of FIGS. 1-8. Computer 1102 may also include one or more network interfaces 1106 for communicating with other devices via a network. Computer 1102 may also include one or more input/output devices 1108 that enable user interaction with computer 1102 (e.g., display, keyboard, mouse, speakers, buttons, etc.).

Processor 1104 may include both general and special purpose microprocessors, and may be the sole processor or one of multiple processors of computer 1102. Processor 1104 may include one or more central processing units (CPUs), for example. Processor 1104, data storage device 1112, and/or memory 1110 may include, be supplemented by, or incorporated in, one or more application-specific integrated circuits (ASICs) and/or one or more field programmable gate arrays (FPGAs).

Data storage device 1112 and memory 1110 each include a tangible non-transitory computer readable storage medium. Data storage device 1112, and memory 1110, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1108 may include peripherals, such as a printer, scanner, display screen, etc. For example, input/output devices 1108 may include a display device such as a cathode ray tube (CRT) or liquid crystal display (LCD) monitor for displaying information to the user, a keyboard, and a pointing device such as a mouse or a trackball by which the user can provide input to computer 1102.

An image acquisition device 1114 can be connected to the computer 1102 to input image data (e.g., medical images) to the computer 1102. It is possible to implement the image acquisition device 1114 and the computer 1102 as one device. It is also possible that the image acquisition device 1114 and the computer 1102 communicate wirelessly through a network. In a possible embodiment, the computer 1102 can be located remotely with respect to the image acquisition device 1114.

Any or all of the systems, apparatuses, and methods discussed herein may be implemented using one or more computers such as computer 1102.

One skilled in the art will recognize that an implementation of an actual computer or computer system may have other structures and may contain other components as well, and that FIG. 11 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving longitudinal medical images of an anatomical object of a patient acquired over a plurality of timepoints; for each respective timepoint of the plurality of timepoints: extracting features from the longitudinal medical images acquired at the respective timepoint using a machine learning based feature extractor network, and analyzing the anatomical object in the longitudinal medical images acquired at the respective timepoint based on the extracted features using a machine learning based prediction model; evaluating progression of the anatomical object over the plurality of timepoints based on results of the analyses using a machine learning based progression model; and outputting the evaluation of the progression of the anatomical object over the plurality of timepoints.

Illustrative embodiment 2. The computer-implemented method of illustrative embodiment 1, wherein the machine learning based progression model receives as input an output of a second-to-last layer of the machine learning based prediction model for each of the plurality of timepoints and generates as output the evaluation of the progression of the anatomical object over the plurality of timepoints.

Illustrative embodiment 3. The computer-implemented method of any one of illustrative embodiments 1-2, wherein the longitudinal medical images comprise patches of the anatomical object extracted from original medical images acquired over the plurality of timepoints, the patches extracted from the original medical images by: extracting features from the original medical images for each of the plurality of timepoints using the machine learning based feature extractor network; segmenting the anatomical object from the original medical images based on the extracted features using a machine learning based segmentation network; and extracting the patches from the original medical images based on the segmentation.

Illustrative embodiment 4. The computer-implemented method of any one of illustrative embodiments 1-3, wherein the machine learning based feature extractor network is trained by: receiving training medical images; degrading the training medical images by applying one or more transformations; extracting training features from the degraded training medical images using the machine learning based feature extractor network; reconstructing the training medical images based on the extracted training features using a machine learning based decoder network; and training the machine learning based feature extractor network and the machine learning based decoder network based on a comparison between the training medical images and reconstructed training medical images.

Illustrative embodiment 5. The computer-implemented method of any one of illustrative embodiments 1-4, wherein the machine learning based feature extractor network is trained using multi-scale training medical images.

Illustrative embodiment 6. The computer-implemented method of any one of illustrative embodiments 1-5, wherein the plurality of timepoints comprises a timepoint corresponding to a baseline examination of the anatomical object, one or more timepoints corresponding to one or more follow-up examinations of the anatomical object, and a timepoint corresponding to a current examination of the anatomical object.

Illustrative embodiment 7. The computer-implemented method of any one of illustrative embodiments 1-6, wherein the anatomical object comprises one or more prostate cancer lesions on a prostate of the patient.

Illustrative embodiment 8. The computer-implemented method of illustrative embodiment 7, wherein the machine learning based progression model is trained to determine at least one of a GGG (Gleason grade group) score or a PIRADS (prostate imaging reporting and data system) score.

Illustrative embodiment 9. The computer-implemented method of any one of illustrative embodiments 1-8, wherein the longitudinal medical images comprise medical images of an MRI (magnetic resonance imaging) sequence.

Illustrative embodiment 10. An apparatus comprising: means for receiving longitudinal medical images of an anatomical object of a patient acquired over a plurality of timepoints; for each respective timepoint of the plurality of timepoints: means for extracting features from the longitudinal medical images acquired at the respective timepoint using a machine learning based feature extractor network, and means for analyzing the anatomical object in the longitudinal medical images acquired at the respective timepoint based on the extracted features using a machine learning based prediction model; means for evaluating progression of the anatomical object over the plurality of timepoints based on results of the analyses using a machine learning based progression model; and means for outputting the evaluation of the progression of the anatomical object over the plurality of timepoints.

Illustrative embodiment 11. The apparatus of illustrative embodiment 10, wherein the machine learning based progression model receives as input an output of a second-to-last layer of the machine learning based prediction model for each of the plurality of timepoints and generates as output the evaluation of the progression of the anatomical object over the plurality of timepoints.

Illustrative embodiment 12. The apparatus of any one of illustrative embodiments 10-11, wherein the longitudinal medical images comprise patches of the anatomical object extracted from original medical images acquired over the plurality of timepoints, the patches extracted from the original medical images by: extracting features from the original medical images for each of the plurality of timepoints using the machine learning based feature extractor network; segmenting the anatomical object from the original medical images based on the extracted features using a machine learning based segmentation network; and extracting the patches from the original medical images based on the segmentation.

Illustrative embodiment 13. The apparatus of any one of illustrative embodiments 10-12, wherein the machine learning based feature extractor network is trained by: receiving training medical images; degrading the training medical images by applying one or more transformations; extracting training features from the degraded training medical images using the machine learning based feature extractor network; reconstructing the training medical images based on the extracted training features using a machine learning based decoder network; and training the machine learning based feature extractor network and the machine learning based decoder network based on a comparison between the training medical images and reconstructed training medical images.

Illustrative embodiment 14. The apparatus of any one of illustrative embodiments 10-13, wherein the machine learning based feature extractor network is trained using multi-scale training medical images.

Illustrative embodiment 15. A non-transitory computer-readable storage medium comprising instructions which, when executed by a computer, cause the computer to carry out operations comprising: receiving longitudinal medical images of an anatomical object of a patient acquired over a plurality of timepoints; for each respective timepoint of the plurality of timepoints: extracting features from the longitudinal medical images acquired at the respective timepoint using a machine learning based feature extractor network, and analyzing the anatomical object in the longitudinal medical images acquired at the respective timepoint based on the extracted features using a machine learning based prediction model; evaluating progression of the anatomical object over the plurality of timepoints based on results of the analyses using a machine learning based progression model; and outputting the evaluation of the progression of the anatomical object over the plurality of timepoints.

Illustrative embodiment 16. The non-transitory computer-readable storage medium of illustrative embodiment 15, wherein the machine learning based progression model receives as input an output of a second-to-last layer of the machine learning based prediction model for each of the plurality of timepoints and generates as output the evaluation of the progression of the anatomical object over the plurality of timepoints.

Illustrative embodiment 17. The non-transitory computer-readable storage medium of any one of illustrative embodiments 15-16, wherein the plurality of timepoints comprises a timepoint corresponding to a baseline examination of the anatomical object, one or more timepoints corresponding to one or more follow-up examinations of the anatomical object, and a timepoint corresponding to a current examination of the anatomical object.

Illustrative embodiment 18. The non-transitory computer-readable storage medium of any one of illustrative embodiments 15-17, wherein the anatomical object comprises one or more prostate cancer lesions on a prostate of the patient.

Illustrative embodiment 19. The non-transitory computer-readable storage medium of illustrative embodiment 18, wherein the machine learning based progression model is trained to determine at least one of a GGG (Gleason grade group) score or a PIRADS (prostate imaging reporting and data system) score.

Illustrative embodiment 20. The non-transitory computer-readable storage medium of any one of illustrative embodiments 15-19, wherein the longitudinal medical images comprise medical images of an MRI (magnetic resonance imaging) sequence.

1. A computer-implemented method comprising:
receiving longitudinal medical images of an anatomical object of a patient acquired over a plurality of timepoints;
for each respective timepoint of the plurality of timepoints:
extracting features from the longitudinal medical images acquired at the respective timepoint using a machine learning based feature extractor network, and
analyzing the anatomical object in the longitudinal medical images acquired at the respective timepoint based on the extracted features using a machine learning based prediction model;
evaluating progression of the anatomical object over the plurality of timepoints based on results of the analyses using a machine learning based progression model; and
outputting the evaluation of the progression of the anatomical object over the plurality of timepoints.

2. The computer-implemented method of claim 1, wherein the machine learning based progression model receives as input an output of a second-to-last layer of the machine learning based prediction model for each of the plurality of timepoints and generates as output the evaluation of the progression of the anatomical object over the plurality of timepoints.

3. The computer-implemented method of claim 1, wherein the longitudinal medical images comprise patches of the anatomical object extracted from original medical images acquired over the plurality of timepoints, the patches extracted from the original medical images by:
extracting features from the original medical images for each of the plurality of timepoints using the machine learning based feature extractor network;
segmenting the anatomical object from the original medical images based on the extracted features using a machine learning based segmentation network; and
extracting the patches from the original medical images based on the segmentation.

4. The computer-implemented method of claim 1, wherein the machine learning based feature extractor network is trained by:
receiving training medical images;
degrading the training medical images by applying one or more transformations;
extracting training features from the degraded training medical images using the machine learning based feature extractor network;
reconstructing the training medical images based on the extracted training features using a machine learning based decoder network; and
training the machine learning based feature extractor network and the machine learning based decoder network based on a comparison between the training medical images and reconstructed training medical images.

5. The computer-implemented method of claim 1, wherein the machine learning based feature extractor network is trained using multi-scale training medical images.

6. The computer-implemented method of claim 1, wherein the plurality of timepoints comprises a timepoint corresponding to a baseline examination of the anatomical object, one or more timepoints corresponding to one or more follow-up examinations of the anatomical object, and a timepoint corresponding to a current examination of the anatomical object.

7. The computer-implemented method of claim 1, wherein the anatomical object comprises one or more prostate cancer lesions on a prostate of the patient.

8. The computer-implemented method of claim 7, wherein the machine learning based progression model is trained to determine at least one of a GGG (Gleason grade group) score or a PIRADS (prostate imaging reporting and data system) score.

9. The computer-implemented method of claim 1, wherein the longitudinal medical images comprise medical images of an MRI (magnetic resonance imaging) sequence.

10. An apparatus comprising:
means for receiving longitudinal medical images of an anatomical object of a patient acquired over a plurality of timepoints;
for each respective timepoint of the plurality of timepoints:
means for extracting features from the longitudinal medical images acquired at the respective timepoint using a machine learning based feature extractor network, and
means for analyzing the anatomical object in the longitudinal medical images acquired at the respective timepoint based on the extracted features using a machine learning based prediction model;
means for evaluating progression of the anatomical object over the plurality of timepoints based on results of the analyses using a machine learning based progression model; and
means for outputting the evaluation of the progression of the anatomical object over the plurality of timepoints.

11. The apparatus of claim 10, wherein the machine learning based progression model receives as input an output of a second-to-last layer of the machine learning based prediction model for each of the plurality of timepoints and generates as output the evaluation of the progression of the anatomical object over the plurality of timepoints.

12. The apparatus of claim 10, wherein the longitudinal medical images comprise patches of the anatomical object extracted from original medical images acquired over the plurality of timepoints, the patches extracted from the original medical images by:
extracting features from the original medical images for each of the plurality of timepoints using the machine learning based feature extractor network;
segmenting the anatomical object from the original medical images based on the extracted features using a machine learning based segmentation network; and
extracting the patches from the original medical images based on the segmentation.

13. The apparatus of claim 10, wherein the machine learning based feature extractor network is trained by:
receiving training medical images;
degrading the training medical images by applying one or more transformations;
extracting training features from the degraded training medical images using the machine learning based feature extractor network;
reconstructing the training medical images based on the extracted training features using a machine learning based decoder network; and
training the machine learning based feature extractor network and the machine learning based decoder network based on a comparison between the training medical images and reconstructed training medical images.

14. The apparatus of claim 10, wherein the machine learning based feature extractor network is trained using multi-scale training medical images.

15. A non-transitory computer-readable storage medium comprising instructions which, when executed by a computer, cause the computer to carry out operations comprising:
receiving longitudinal medical images of an anatomical object of a patient acquired over a plurality of timepoints;
for each respective timepoint of the plurality of timepoints:
extracting features from the longitudinal medical images acquired at the respective timepoint using a machine learning based feature extractor network, and
analyzing the anatomical object in the longitudinal medical images acquired at the respective timepoint based on the extracted features using a machine learning based prediction model;
evaluating progression of the anatomical object over the plurality of timepoints based on results of the analyses using a machine learning based progression model; and
outputting the evaluation of the progression of the anatomical object over the plurality of timepoints.

16. The non-transitory computer-readable storage medium of claim 15, wherein the machine learning based progression model receives as input an output of a second-to-last layer of the machine learning based prediction model for each of the plurality of timepoints and generates as output the evaluation of the progression of the anatomical object over the plurality of timepoints.

17. The non-transitory computer-readable storage medium of claim 15, wherein the plurality of timepoints comprises a timepoint corresponding to a baseline examination of the anatomical object, one or more timepoints corresponding to one or more follow-up examinations of the anatomical object, and a timepoint corresponding to a current examination of the anatomical object.

18. The non-transitory computer-readable storage medium of claim 15, wherein the anatomical object comprises one or more prostate cancer lesions on a prostate of the patient.

19. The non-transitory computer-readable storage medium of claim 18, wherein the machine learning based progression model is trained to determine at least one of a GGG (Gleason grade group) score or a PIRADS (prostate imaging reporting and data system) score.

20. The non-transitory computer-readable storage medium of claim 15, wherein the longitudinal medical images comprise medical images of an MRI (magnetic resonance imaging) sequence.
